(19) United States
(12) Patent Application Publication
(10) Pub. No.: US 2015/0125546 A1
(43) Pub. Date: May 7, 2015

(54) COMBINATION THERAPY FOR TREATING PULMONARY HYPERTENSION

(71) Applicant: Gilead Sciences, Inc., Foster City, CA (US)

(72) Inventors: Luiz Belardinelli, Palo Alto, CA (US); Hunter Campbell Gillies, El Granada, CA (US); Faquan Liang, San Francisco, CA (US); Lina Yao, San Mateo, CA (US)

(73) Assignee: Gilead Sciences, Inc., Foster City, CA (US)

(21) Appl. No.: 14/534,396

(22) Filed: Nov. 6, 2014

Related U.S. Application Data
(60) Provisional application No. 61/988,120, filed on May 2, 2014.
(60) Provisional application No. 61/900,863, filed on Nov. 6, 2013.

(51) Int. Cl. A61K 31/506; A61K 45/06; A61K 31/505

(52) U.S. Cl. CPC A61K 31/506; A61K 31/505; A61K 45/06
USPC 424/613; 514/274

(57) ABSTRACT

Method of treating and/or preventing pulmonary arterial hypertension comprising administering effective amounts of ambrisentan or a pharmaceutically acceptable salt thereof and riociguat or a pharmaceutically acceptable salt thereof.

### CROSS-REFERENCE TO RELATED APPLICATIONS

This application claims benefit under 35 U.S.C. §119(e) to U.S. Provisional Application Ser. No. 61/900,863, filed on Nov. 6, 2013 and Provisional Application Ser. No. 61/988,120, filed May 2, 2014, the entirety of which are incorporated herein by reference.

### BACKGROUND

Pulmonary hypertension (PH) has been previously classified as primary (idiopathic) or secondary. Recently, the World Health Organization (WHO) has classified pulmonary hypertension into five groups: Group 1: pulmonary arterial hypertension (PAH); Group 2: PH with left heart disease; Group 3: PH with lung disease and/or hypoxemia; Group 4: PH due to chronic thrombotic and/or embolic disease; and Group 5: miscellaneous conditions (for example, sarcoidosis, histiocytosis X, lymphangiomatosis and compression of pulmonary vessels). See, for example, Rubin (2004) Chest 126:7-10.

Pulmonary arterial hypertension (PAH) is a particular type of PH and is a serious, progressive and life-threatening disease of the pulmonary vasculature, characterized by profound vasoconstriction and an abnormal proliferation of smooth muscle cells in the walls of the pulmonary arteries. Severe constriction of the blood vessels in the lungs leads to very high pulmonary arterial pressures. These high pressures make it difficult for the heart to pump blood through the lungs to be oxygenated. Patients with PAH suffer from extreme shortness of breath as the heart struggles to pump against these high pressures. Patients with PAH typically develop significant increases in pulmonary vascular resistance (PVR) and sustained elevations in pulmonary artery pressure (PAP), which ultimately lead to right ventricular failure and death. Patients diagnosed with PAH have a poor prognosis and equally compromised quality of life, with a mean life expectancy of 2 to 5 years from the time of diagnosis if untreated.

Endothelin-1 (ET-1) is the primary member of a family of potent vasoconstrictor peptides, which are known to play an essential role in mammalian cardiovascular physiology. ET-1 is synthesized de novo and released from endothelial cells in response to a variety of factors, including angiotensin II, catecholamines, cytokines, hypoxia and shear stress. Two receptor subtypes, endothelin receptor type A (ET_{A}) and endothelin receptor type B (ET_{B}), mediate the effects of ET-1. In humans, the ET_{A} receptor is preferentially expressed in vascular smooth muscle cells and is primarily responsible for the vasoconstrictive effects of ET-1. In contrast, ET_{B} receptors are found mainly in the vascular endothelium, and their activation results in vasodilatation via production of nitric oxide and prostacyclin. The ET_{B} receptor is also involved in regulation of circulating concentrations of ET-1, through effects on endothelin converting enzyme (ECE-1) expression, and the synthesis and reuptake of ET-1 by endothelial cells.

Ambrisentan is a non-sulfonamide, propanoic acid-class endothelin receptor antagonist (ERA) with high affinity (about 12 pM) for the ET_{A} receptor. Ambrisentan is approved for sale by the U.S. Food and Drug Administration (FDA) for once-daily treatment of PAH and is marketed under the trade name Letairis®. Other selective type-A receptor antagonists include sitaxentan, atrasentan, and BQ-123.

U.S. Patent Publication No. 2008/0139593 describes a method for treating pulmonary hypertension, comprising administration of an effective amount of ambrisentan to a patient, wherein, at baseline, time from the first diagnosis of the condition in the subject is not greater than about two years. Also described in the publication is ambrisentan in combination with one or more suitable drugs selected from prostanoids, PDE5 inhibitors such as sildenafil, tadalafil, and vardenafil, ERAs, calcium channel blockers, arylalkylamines, dihydropyridine derivatives, piperazine derivatives and other suitable compounds for use in combination therapy.

U.S. Patent Publication No. 2012/0269898 describes a method for treating pulmonary arterial hypertension, comprising administration of an effective amount of ambrisentan in combination with a PDE5 inhibitor.

Riociguat, a stimulator of soluble guanylate cyclase, was recently approved for treatment of PAH. The known treatments for PAH notwithstanding, there is a need to provide improved treatment modalities for patients suffering from PAH.

### SUMMARY

It has been observed that the combination of ambrisentan and riociguat acts to inhibit endothelin-induced contraction of pulmonary arteries in a more than additive manner. When administering an effective amount of ambrisentan or a pharmaceutically acceptable salt thereof in combination with an effective amount of riociguat or a pharmaceutically acceptable salt thereof, wherein the weight ratio of ambrisentan or a pharmaceutically acceptable salt thereof to riociguat or a pharmaceutically acceptable salt thereof is in a range from about 1:1 to about 1:10 or from about 1:1 to about 10:1, a more than additive effect on inhibit endothelin-induced contraction of pulmonary arteries.

In one aspect, is provided a pharmaceutical composition comprising an effective amount of ambrisentan or a pharmaceutically acceptable salt thereof in combination with an effective amount of riociguat or a pharmaceutically acceptable salt thereof, wherein the weight ratio of ambrisentan or a pharmaceutically acceptable salt thereof to riociguat or a pharmaceutically acceptable salt thereof is in a range from about 1:1 to about 1:10 or from about 1:1 to about 100:1.

In one aspect, this disclosure is directed to a method for treatment and/or prevention of pulmonary hypertension arterial in a patient in need thereof, said method comprising administering to the patient an effective amount of ambrisentan or a pharmaceutically acceptable salt thereof in combination with an effective amount of riociguat or a pharmaceutically acceptable salt thereof.

In another aspect, this disclosure is directed to a method for treatment and/or prevention of pulmonary arterial hypertension in a patient in need thereof, said method comprising administering to the patient an effective amount of ambrisentan or a pharmaceutically acceptable salt thereof in combination with an effective amount of riociguat or a pharmaceutically acceptable salt thereof, wherein the weight ratio of ambrisentan or a pharmaceutically acceptable salt thereof to riociguat or a pharmaceutically acceptable salt thereof is in a range from about 1:1 to about 1:10 or from about 1:1 to about 10:1.

In another aspect, provided is a method for inhibiting endothelin-induced vasoconstriction in a patient in need thereof, said method comprising administering to the patient an effective amount of ambrisentan or a pharmaceutically acceptable salt thereof in combination with an effective amount of riociguat or a pharmaceutically acceptable salt thereof.

In another aspect, provided is a method for inhibiting endothelin-induced vasoconstriction in a patient in need thereof comprising said method comprising administering to the patient an effective amount of ambrisentan or a pharmaceutically acceptable salt thereof in combination with an effective amount of riociguat or a pharmaceutically acceptable salt thereof, wherein the weight ratio of ambrisentan or a pharmaceutically acceptable salt thereof to riociguat or a pharmaceutically acceptable salt thereof is in a range from about 1:1 to about 1:10 or from about 1:1 to about 10:1.

Also provided, in one aspect, is a method for treating or preventing a disease in a patient in need thereof comprising administering to the patient an effective amount of ambrisentan or a pharmaceutically acceptable salt thereof in combination with an effective amount of riociguat or a pharmaceutically acceptable salt thereof, wherein the disease is selected from the group consisting of hypertension, pulmonary hypertension, pulmonary arterial hypertension, myocardial infarction, angina pectoris, acute kidney failure, renal insufficiency, cerebral vasospasms, cerebral ischemia, subarachnoid hemorrhages, asthma, atherosclerosis, intravascular coagulation, restenosis after angioplasty, hypertension caused by ischemia or intoxication, kidney failure caused by ischemia or intoxication, Raynaud's syndrome and asthmatic airway condition.

Also provided, in one aspect, is a method for treating or preventing a disease in a patient in need thereof comprising administering to the patient an effective amount of ambrisentan or a pharmaceutically acceptable salt thereof in combination with an effective amount of riociguat or a pharmaceutically acceptable salt thereof, wherein the disease is selected from the group consisting of hypertension, pulmonary hypertension, myocardial infarction, angina pectoris, acute kidney failure, renal insufficiency, cerebral vasospasms, cerebral ischemia, subarachnoid hemorrhages, asthma, atherosclerosis, intravascular coagulation, restenosis after angioplasty, hypertension caused by ischemia or intoxication, kidney failure caused by ischemia or intoxication, Raynaud's syndrome and asthmatic airway condition, wherein the weight ratio of ambrisentan or a pharmaceutically acceptable salt thereof to riociguat or a pharmaceutically acceptable salt thereof is in a range from about 1:1 to about 1:10 or from about 1:1 to about 10:1.

Also provided, in one aspect, is a method for reducing an effective dose of ambrisentan or a pharmaceutically acceptable salt thereof, the method comprising administering to the patient an effective amount of riociguat or a pharmaceutically acceptable salt thereof, wherein the effectiveness of administration of ambrisentan or a pharmaceutically acceptable salt thereof to riociguat or a pharmaceutically acceptable salt thereof is at least about 25% greater than the sum of effectiveness of mono-administrations of ambrisentan or a pharmaceutically acceptable salt thereof to riociguat or a pharmaceutically acceptable salt thereof.

Also provided, in one aspect, is a method for reducing an effective dose of ambrisentan or a pharmaceutically acceptable salt thereof, the method comprising administering to the patient an effective amount of riociguat or a pharmaceutically acceptable salt thereof, wherein (a) the weight ratio of ambrisentan or a pharmaceutically acceptable salt thereof to riociguat or a pharmaceutically acceptable salt thereof is in a range of from about 1:1 to about 1:10 or from about 1:1 to about 10:1 and/or (b) the effectiveness of administration of ambrisentan or a pharmaceutically acceptable salt thereof to riociguat or a pharmaceutically acceptable salt thereof is at least about 25% greater than the sum of effectiveness of mono-administrations of ambrisentan or a pharmaceutically acceptable salt thereof to riociguat or a pharmaceutically acceptable salt thereof.

In another aspect, provided is a method for reducing an effective dose of riociguat or a pharmaceutically acceptable salt thereof comprising administering to the patient an effective amount of ambrisentan or a pharmaceutically acceptable salt thereof, wherein the effectiveness of administration of ambrisentan or a pharmaceutically acceptable salt thereof to riociguat or a pharmaceutically acceptable salt thereof is at least about 25% greater than the sum of effectiveness of mono-administrations of the ambrisentan or a pharmaceutically acceptable salt thereof to riociguat or a pharmaceutically acceptable salt thereof.

In another aspect, provided is a method for reducing an effective dose of riociguat or a pharmaceutically acceptable salt thereof comprising administering to the patient an effective amount of ambrisentan or a pharmaceutically acceptable salt thereof, wherein (a) the weight ratio of ambrisentan or a pharmaceutically acceptable salt thereof to riociguat or a pharmaceutically acceptable salt thereof is in a range 1:1 to about 1:10 or from about 1:1 to about 10:1 and/or (b) the effectiveness of administration of ambrisentan or a pharmaceutically acceptable salt thereof to riociguat or a pharmaceutically acceptable salt thereof is at least about 25% greater than the sum of effectiveness of mono-administrations of the ambrisentan or a pharmaceutically acceptable salt thereof to riociguat or a pharmaceutically acceptable salt thereof.

In another aspect, provided is a pharmaceutical formulation comprising an effective amount of ambrisentan or a pharmaceutically acceptable salt thereof in combination with an effective amount of riociguat or a pharmaceutically acceptable salt thereof, wherein the effectiveness of combination of ambrisentan or a pharmaceutically acceptable salt thereof to riociguat or a pharmaceutically acceptable salt thereof is at least about 25% greater than the sum of effectiveness of mono-administrations of ambrisentan or a pharmaceutically acceptable salt thereof to riociguat or a pharmaceutically acceptable salt thereof.

In another aspect, provided is a pharmaceutical formulation comprising an effective amount of ambrisentan or a pharmaceutically acceptable salt thereof in combination with an effective amount of riociguat or a pharmaceutically acceptable salt thereof, wherein the weight ratio of ambrisentan or a pharmaceutically acceptable salt thereof to riociguat or a pharmaceutically acceptable salt thereof is in a range 1:1 to about 1:10 or from about 1:1 to about 10:1 and/or (b) the effectiveness of combination of ambrisentan or a pharmaceutically acceptable salt thereof to riociguat or a pharmaceutically acceptable salt thereof is at least about 25% greater than the sum of effectiveness of mono-administrations of ambrisentan or a pharmaceutically acceptable salt thereof to riociguat or a pharmaceutically acceptable salt thereof.

In another aspect is provided a method for the manufacture of a medicament comprising an effective amount of ambrisentan or a pharmaceutically acceptable salt thereof in combination with an effective amount of riociguat or a pharmaceutically acceptable salt thereof, wherein the weight ratio of ambrisentan or a pharmaceutically acceptable salt thereof to riociguat or a pharmaceutically acceptable salt thereof is in a range 1:1 to about 1:10 or from about 1:1 to about 10:1.

In another aspect is provided a method for the manufacture of a medicament comprising a fixed dose combination of an effective amount of ambrisentan or a pharmaceutically acceptable salt thereof with an effective amount of riociguat or a pharmaceutically acceptable salt thereof, wherein the weight ratio of ambrisentan or a pharmaceutically acceptable salt thereof to riociguat or a pharmaceutically acceptable salt thereof is in a range 1:1 to about 1:10 or from about 1:1 to about 10:1. In one embodiment, the fixed dose combination is a tablet.

In another aspect is provided a fixed dose combination of an effective amount of ambrisentan or a pharmaceutically acceptable salt thereof in combination with an effective amount of riociguat or a pharmaceutically acceptable salt thereof, wherein the weight ratio of ambrisentan or a pharmaceutically acceptable salt thereof to riociguat or a pharmaceutically acceptable salt thereof is in a range 1:1 to about 1:10 or from about 1:1 to about 10:1 for use in therapy.

### BRIEF DESCRIPTION OF THE DRAWINGS

FIG. 1 shows concentration-dependent effects of ambrisentan and riociguat on ET-1-induced contraction of rat pulmonary arteries.

FIG. 2 shows that ambrisentan (10 nM) and riociguat (30 nM), in combination, relaxed endothelin-induced contraction of rat pulmonary arteries significantly more than combination of bosentan (100 nM) and riociguat (30 nM) or combination of macitentan (30 nM) and riociguat (30 nM) or mono-administration of any of the drugs. *p<0.01 versus mono-therapy of 10 nM Ambrisentan or 30 nM riociguat. #p<0.05 vs. mono-therapy of 100 nM bosentan, 30 nM macitentan, 30 nM riociguat, or combination of 10 nM ambrisentan and 30 nM riociguat.

FIG. 3 shows the effects of ambrisentan in combination with riociguat on ET-1-induced contraction of rat pulmonary arteries.

FIG. 4 shows the additive effect of macitentan or bosentan in combination with riociguat on ET-1-induced contraction of rat pulmonary arteries.

FIG. 5 shows that endothelial ET_{B} receptor contributes to the effect of ambrisentan and riociguat on pulmonary vaso-relaxation.

FIG. 6 shows that riociguat increases potency of ambrisentan to relax ET-1 constricted rat pulmonary arteries.

FIG. 7 shows that ambrisentan increases potency of riociguat to relax ET-1 constricted rat pulmonary arteries.

FIG. 8 shows that riociguat potentiated ET-1-stimulated cGMP production in rat intrapulmonary arterial rings.

FIG. 9 shows that bosentan blocked ET-1-stimulated cGMP production and impaired the effect of ET-1 and riociguat on cGMP production.

FIG. 10 shows that ambrisentan and riociguat attenuated ET-1-stimulated phosphorylation of MLC2 by MLCK in rat intrapulmonary arteries.

FIG. 11 shows mechanism of action of ambrisentan in combination with riociguat against ET-induced pulmonary vasoconstriction.

### DETAILED DESCRIPTION

### Definitions

As used in the present specification, the following words and phrases are generally intended to have the meanings as set forth below, except to the extent that the context in which they are used indicates otherwise.

It is to be noted that as used herein and in the claims, the singular forms “a,” “an” and “the” include plural referents unless the context clearly dictates otherwise. Thus, for example, reference to “a pharmaceutically acceptable carrier” in a composition includes two or more pharmaceutically acceptable carriers, and so forth.

“Comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

An “endothelin receptor antagonist (ERA)” is an agent that blocks endothelin receptors. There are at least two major known endothelin receptors, ET_{A} and ET_{B}, both of which are G protein-coupled receptors whose activation result in elevation of intracellular-free calcium. Three main kinds of ERAs exist: selective type-A receptor antagonists, e.g., sitaxentan, ambrisentan, atrasentan, BQ-123, which affect endothelin A receptors; dual antagonists, e.g., bosentan, macitentan, tezosentan, which affect both endothelin A and B receptors; and selective type-B receptor antagonists, e.g., BQ-788, which affect endothelin B receptors.

A “selective type-A endothelin receptor antagonist” or “selective type-A ERA” selectively targets the type-A endothelin receptor.

“Ambrisentan” or “AMB” is described in U.S. Pat. Nos. 5,703,017; 5,932,730 and 7,109,205. It refers to the chemical compound, (2S)-2-[(4,6-dimethylpyrimidin-2-yl)oxy]-3-methoxy-3,3-diphenylpropanoic acid and has the following chemical formula:

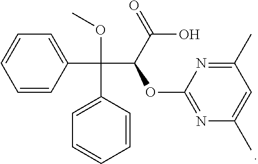 [SMILES: COC(c1ccccc1)(c1ccccc1)[C@H](Oc1nc(C)cc(C)n1)C(=O)O]

Ambrisentan is approved for sale by the U.S. Food and Drug Administration (FDA) for once-daily treatment of PAH and is marketed under the trade name Letairis®. In Europe, Ambrisentan is approved under the trade name Volibris®.

“Ambrisentan” as used herein is intended to include the metabolites of ambrisentan described in U.S. Patent Publication No. 2010/0204163. The ambrisentan metabolites include the compounds having the following chemical formula:

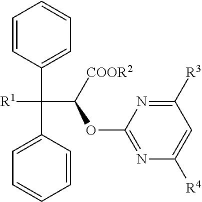

wherein R¹ is —OH or —OCH₃; R² is —H, lower alkyl or glycosidyl; and R³ and R⁴ are independently —CH₃, —C(O)H or —CH₂OR⁶, wherein R⁶ is —H or a hydrocarbyl group having 1 to 20 carbon atoms.

“Ambrisentan” as used herein is also intended to include a pharmaceutically acceptable salt thereof.

Riociguat is approved for sale by the U.S. Food and Drug Administration (FDA) for treatment of PAH or chronic thromboembolic pulmonary hypertension and is marketed under the trade name Adempas®.

“Riociguat” is described in U.S. Pat. No. 7,171,037. It refers to the chemical compound, methyl (4,6-diamino-2-(1-(2-fluorobenzyl)-1H-pyrazolo[3,4-b]pyridin-3-yl)pyrimidin-5-yl)(methyl)carbamate and has the following chemical formula:

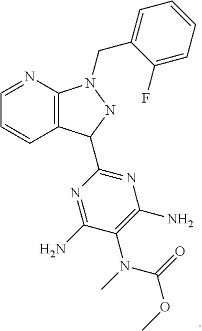 [SMILES: COC(=O)N(C)c1c(N)nc(C2NN(Cc3ccccc3F)c3ncccc32)nc1N]

As used herein, the term “salt” refers to a “pharmaceutically acceptable salt” refers to a salt of a compound that is derived from a variety of physiologically acceptable organic and inorganic counter ions useful for pharmaceutical purposes. Such counter ions are well known in the art and include, by way of example only, sodium, potassium, calcium, magnesium, aluminum, lithium and ammonium, for example tetraalkylammonium, and the like when the molecule contains an acidic functionality; and when the molecule contains a basic functionality, salts of organic or inorganic acids, such as hydrochloride, sulfate, phosphate, diphosphate, nitrate hydrobromide, tartrate, mesylate, acetate, malate, maleate, besylate, fumarate, tartrate, succinate, citrate, lactate, pamoate, salicylate, stearate, methanesulfonate, p-toluenesulfonate, and oxalate, and the like. Suitable pharmaceutically acceptable salts also include those listed in Remington's Pharmaceutical Sciences, 17th Edition, pg. 1418 (1985) and P. Heinrich Stahl, Camille G. Wermuth (Eds.), Handbook of Pharmaceutical Salts Properties, Selection, and Use; 2002. Examples of acid addition salts include those formed from acids such as hydroiodic, phosphoric, metaphosphoric, nitric and sulfuric acids, and with organic acids, such as alginic, ascorbic, anthranilic, benzoic, camphorsulfuric, citric, embonic (pamoic), ethanesulfonic, formic, fumaric, furoic, galacturonic, gentisic, gluconic, glucuronic, glutamic, glycolic, isonicotinic, isothionic, lactic, malic, mandelic, methanesulfonic, mucic, pantothenic, phenylacetic, propionic, saccharic, salicylic, stearic, succinic, sulfinilic, trifluoroacetic and arylsulfonic for example benzenesulfonic and p-toluenesulfonic acids. Examples of base addition salts formed with alkali metals and alkaline earth metals and organic bases include chloroprocaine, choline, N,N-dibenzylethylenediamine, diethanolamine, ethylenediamine, lysine, meglumaine (N-methylglucamine), and procaine, as well as internally formed salts. Salts having a non-physiologically acceptable anion or cation are within the scope of the disclosure as useful intermediates for the preparation of physiologically acceptable salts and/or for use in non-therapeutic, for example, in vitro, situations.

The disclosure contemplates using the base forms or salt forms of both ambrisentan and riociguat and further contemplates mixtures of salts of riociguat and/or ambrisentan.

In certain embodiments, the ambrisentan and/or riociguat as used herein has not been sufficiently ionized and may be in the form a co-crystal. In one embodiment, the present disclosure provides a co-crystal composition comprising a co-crystal of ambrisentan and/or riociguat, wherein said co-crystal comprises ambrisentan and/or riociguat and a co-crystal former. The term “co-crystal” refers a crystalline material which comprises ambrisentan and/or riociguat and one or more co-crystal formers, such as a pharmaceutically acceptable salt. In certain embodiments, the co-crystal can have an improved property as compared to the free form (i.e., the free molecule, zwitter ion, hydrate, solvate, etc.) or a salt (which includes salt hydrates and solvates). In further embodiments, the improved property is selected from the group consisting of: increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like. Methods for making and characterizing co-crystals are well known to those of skill in the art.

The phrase “combination therapy,” in defining use of a selective type-A endothelin receptor antagonist and a stimulator of soluble guanylate cyclase, is intended to embrace administration of each agent in a sequential manner in a regimen that will provide beneficial effects of the drug combination, and is intended as well to embrace co-administration of these agents in a substantially simultaneous manner, such as by oral ingestion of a single tablet or capsule having a fixed ratio of these active agents or ingestion of multiple, separate tablets or capsules for each agent. “Combination therapy” will also include simultaneous or sequential administration by intravenous, intramuscular or other parenteral routes into the body, including direct absorption through mucous membrane tissues, as found in the sinus passages. Sequential administration also includes drug combination where the individual elements may be administered at different times and/or by different routes but which act in combination to provide a beneficial effect, such as enhanced effectiveness.

The phrases “fixed dosage combination” and “single dosage form” are synonymous and each means a pharmaceutical formulation comprising two or more active ingredients wherein the dose or amount of each component is fixed and all components are presented in a single formulation such as a single tablet, capsule, volume of parenteral formulation or other formulation disclosed herein.

In one embodiment, a combination therapy consists essentially of two active agents, namely, the selective type-A endothelin receptor antagonist, ambrisentan and the stimulator of soluble guanylate cyclase, riociguat.

In another embodiment, a combination therapy is a three-way combination comprising a selective type-A endothelin receptor antagonist, a stimulator of soluble guanylate cyclase and a third active agent effective for the treatment of the pulmonary arterial hypertension condition or a condition related thereto. Illustratively and without limitation, the combination can include a third active agent selected from the group consisting of prostanoids, endothelin receptor antagonists other than ambrisentan, calcium channel blockers, diuretics, anticoagulants, a stimulator of soluble guanylate cyclase other than riociguat, oxygen and combinations thereof.

The phrase “therapeutically-effective” is intended to qualify the amount of each agent for use in the combination therapy which will achieve the goal of improvement in pulmonary functions, while avoiding or reducing an adverse side effect typically associated with each agent. The therapeutically effective amount will vary depending upon the specific activity of the therapeutic agent being used, the severity of the patient's disease state, the age, physical condition, existence of other disease states, and nutritional status of the patient. Additionally, other medication the patient may be receiving will affect the determination of the therapeutically effective amount of the therapeutic agent to administer.

“Co-action” or “synergistically” means that the therapeutic effect of a stimulator of soluble guanylate cyclase such as riociguat, when administered in combination with a selective type-A endothelin receptor antagonist such as ambrisentan (or vice-versa) is greater than the sum of the therapeutic effects of the agents when administered separately. The term “therapeutic amount” or “therapeutically effective amount” used herein includes a less than standard therapeutic amount of one or both drugs, meaning that the amount required for the desired effect is lower than when the drug is used separately. As used herein, an effective amount also means when one drug is administered or co-administered at a higher dose than is standard while the other drug is administered at an equal or lower dose than standard. A therapeutic amount also includes when one drug is given at a standard therapeutic dose and another drug is administered in a less than standard therapeutic dose. For example, ambrisentan could be given in a therapeutic dose and riociguat could be given in a less than standard therapeutic dose to provide an enhanced result. In some embodiments, both drugs can be administered in a standard therapeutic dose for much greater efficacies.

The term “treatment” or “treating” means any treatment of a disease or condition in a subject, such as a mammal, including: 1) preventing or protecting against the disease or condition, that is, causing the clinical symptoms not to develop; 2) inhibiting the disease or condition, that is, arresting or suppressing the development of clinical symptoms; and/or 3) relieving the disease or condition that is, causing the regression of clinical symptoms. In one embodiment, the mammal is a human.

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment.

It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.”

The term “patient” typically refers to a “mammal” which includes, without limitation, humans, monkeys, rabbits, mice, domestic animals, such as dogs and cats, farm animals, such as cows, horses, or pigs, and laboratory animals.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Intravenous administration” is the administration of substances directly into a vein, or “intravenously.” Compared with other routes of administration, the intravenous (N) route is the fastest way to deliver fluids and medications throughout the body. An infusion pump can allow precise control over the flow rate and total amount delivered, but in cases where a change in the flow rate would not have serious consequences, or if pumps are not available, the drip is often left to flow simply by placing the bag above the level of the patient and using the clamp to regulate the rate. Alternatively, a rapid infuser can be used if the patient requires a high flow rate and the IV access device is of a large enough diameter to accommodate it. This is either an inflatable cuff placed around the fluid bag to force the fluid into the patient or a similar electrical device that may also heat the fluid being infused. When a patient requires medications only at certain times, intermittent infusion is used, which does not require additional fluid. It can use the same techniques as an intravenous drip (pump or gravity drip), but after the complete dose of medication has been given, the tubing is disconnected from the IV access device. Some medications are also given by IV push or bolus, meaning that a syringe is connected to the IV access device and the medication is injected directly (slowly, if it might irritate the vein or cause a too-rapid effect). Once a medicine has been injected into the fluid stream of the IV tubing there must be some means of ensuring that it gets from the tubing to the patient. Usually this is accomplished by allowing the fluid stream to flow normally and thereby carry the medicine into the bloodstream; however, a second fluid injection is sometimes used, a “flush,” following the injection to push the medicine into the bloodstream more quickly.

“Oral administration” is a route of administration where a substance is taken through the mouth, and includes buccal, sublabial and sublingual administration, as well as enteral administration and that through the respiratory tract, unless made through, e.g., tubing so the medication is not in direct contact with any of the oral mucosa. Typical foam for the oral administration of therapeutic agents includes the use of tablets or capsules.

### Methods

This disclosure describes the administration of a selective type-A endothelin receptor antagonist in combination with a stimulator of soluble guanylate cyclase which co-acts in relaxing pulmonary contractions and/or inhibiting hypoxia-induced pulmonary arterial pressure (PAP). The ability to relax pulmonary contraction or inhibit PAP is useful for treating and preventing pulmonary hypertension in patients, as well as a variety of other conditions, which are described herein. This combination therapy leads to enhanced therapeutic effects when the selective type-A ERA is administered in an effective dose and the stimulator of soluble guanylate cyclase is administered in an effective dose. Either one or both of the selective type-A ERA and the stimulator of soluble guanylate cyclase may be administered in an amount less than their respective standard therapeutic doses due to their co-action.

Certain ratios of the two agents even increase effectiveness of the co-action so that it is substantially greater than the sum of effectiveness of mono-administration of each agent (i.e., administration of a single agent). In one aspect, the ratio of the amount of the selective type-A ERA and the amount of the stimulator of soluble guanylate cyclase, in order to get such enhanced effectiveness, can be 1:1 to about 1:10 or from about 1:1 to about 10:1. In one embodiment, the ratio of the amount of the selective type-A ERA and the amount of the stimulator of soluble guanylate cyclase can range from about 1:1, about 1:1.5, about 1:2, about 1:2.5, about 1:3, about 1:4, about 1:5, about 1:6, about 1:6.5, about 1:7, about 1:7.5, about 1:8, about 1:8.5, about 1:9, about 1:9.5, or about 1:10. In another embodiment, the ratio of the amount of the selective type-A ERA and the amount of the stimulator of soluble guanylate cyclase can range from about 10.0:1, about 9.5:1, about 9.0:1, about 8.5:1, about 8.0:1, about 7.5:1, about 7.0:1, about 6.5:1, about 6.0:1, about 5.5:1, about 5.0:1, about 4.5:1, about 4.0:1, about 3.5:1, about 3.0:1, or about 2.5:1. In another aspect, such combinations can achieve an effectiveness that is at least about 5%, or alternatively about 10%, about 15%, about 20%, about 25%, about 30%, about 35%, about 40%, about 45%, about 50%, about 55%, about 60%, about 65%, about 70%, about 80%, about 90% or about 100% greater than the sum of effectiveness of mono-administration of each agent.

In one embodiment the weight ration of ambrisentan or a pharmaceutically acceptable salt thereof to riociguat or a pharmaceutically acceptable salt thereof is in a range about 10:1. In another embodiment, the weight ratio of ambrisentan or a pharmaceutically acceptable salt thereof to riociguat or a pharmaceutically acceptable salt thereof is about 8:1. In yet another embodiment the weight ration of ambrisentan or a pharmaceutically acceptable salt thereof to riociguat or a pharmaceutically acceptable salt thereof is in a range about 5:1.

In one aspect, the ratio of amounts is a ratio of molar amounts of each agent. In another aspect, the ratio of amounts is a weight ratio of each agent.

Accordingly, in one aspect, this disclosure is directed to a method for treatment and/or prevention of pulmonary hypertension in a patient in need thereof, said method comprising administering to the patient an effective amount of ambrisentan or a pharmaceutically acceptable salt thereof in combination with an effective amount of riociguat or a pharmaceutically acceptable salt thereof. In some embodiments, said method comprises the weight ratio of ambrisentan or a pharmaceutically acceptable salt thereof to riociguat or a pharmaceutically acceptable salt thereof in a range from about 10:1.

In other embodiments, said method comprises the weight ratio of ambrisentan or a pharmaceutically acceptable salt thereof to riociguat or a pharmaceutically acceptable salt thereof in a range from about 1:1 to about 1:10 or from about 1:1 to 10:1; and wherein ambrisentan or a pharmaceutically acceptable salt thereof to riociguat or a pharmaceutically acceptable salt thereof are formulated separately, or alternatively in a single dosage form.

In other embodiments, said method comprises the weight ratio of ambrisentan or a pharmaceutically acceptable salt thereof to riociguat or a pharmaceutically acceptable salt thereof in a range from about 1:1 to about 1:10 or from about 1:1 to 10:1, and wherein ambrisentan or a pharmaceutically acceptable salt thereof to riociguat or a pharmaceutically acceptable salt thereof are formulated for parenteral administration, or alternatively for oral administration.

In other embodiments, said method comprises the weight ratio of ambrisentan or a pharmaceutically acceptable salt thereof to riociguat or a pharmaceutically acceptable salt thereof is in a range from about 1:1 to about 1:10 or from about 1:1 to 10:1, and wherein ambrisentan or a pharmaceutically acceptable salt thereof to riociguat or a pharmaceutically acceptable salt thereof are formulated in tablet form or capsule form.

In other embodiments, said method comprises the weight ratio of ambrisentan or a pharmaceutically acceptable salt thereof to riociguat or a pharmaceutically acceptable salt thereof in a range from about 1:1 to about 1:10 or from about 1:1 to 10:1, and wherein ambrisentan or a pharmaceutically acceptable salt thereof to riociguat or a pharmaceutically acceptable salt thereof are administered once daily.

In other embodiments, said method comprises the weight ratio of ambrisentan or a pharmaceutically acceptable salt thereof to riociguat or a pharmaceutically acceptable salt thereof in a range from about 1:1 to about 1:10, and wherein the amount of ambrisentan or a pharmaceutically acceptable salt thereof is from about 0.1 mg to about 100 mg daily, or alternatively from about 2 mg to about 20 mg daily, and wherein the amount of riociguat or a pharmaceutically acceptable salt thereof administered is from about 0.1 mg to about 600 mg daily, or alternatively from about 0.2 mg to about 2.0 mg daily or alternatively from about 0.2 mg to about 120 mg daily.

In other embodiments, said method comprises the weight ratio of ambrisentan or a pharmaceutically acceptable salt thereof to riociguat or a pharmaceutically acceptable salt thereof is in a range from about 1:1 to about 1:10 or from about 1:1 to 10:1, the method further comprising administering a third active agent comprising at least one drug selected from the group consisting of prostanoids, endothelin receptor antagonists other than ambrisentan, calcium channel blockers, diuretics, anticoagulants, a stimulator of soluble guanylate cyclase other than riociguat, oxygen and combinations thereof.

In any of the embodiments described herein, pulmonary arterial hypertension (PAH), includes but is not limited to idiopathic PAH, familial PAH or PAH associated with another disease or condition. In one aspect, the PAH at baseline is of WHO Class I, II, III or IV.

In another aspect, provided is a method for inhibiting endothelin-induced vasoconstriction in a patient in need thereof comprising said method comprising administering to the patient an effective amount of ambrisentan or a pharmaceutically acceptable salt thereof in combination with an effective amount of riociguat or a pharmaceutically acceptable salt thereof, wherein the weight ratio of ambrisentan or a pharmaceutically acceptable salt thereof to riociguat or a pharmaceutically acceptable salt thereof is in a range from about 1:1 to about 1:10 or from about 1:1 to 10:1.

In another aspect, is provided a method of treating pulmonary arterial hypertension comprising co-administering an effective dose of ambrisentan or a pharmaceutically acceptable salt thereof to riociguat or a pharmaceutically acceptable salt thereof to a patient in need thereof. In one embodiment, the dose of ambrisentan 5 mg or 10 mg and the dose of riociguat is selected from the group consisting of 0.5 mg, 1.0 mg, 1.5 mg, 2.0 mg and 2.5 mg. In yet another aspect is provided a method of treating pulmonary arterial hypertension comprising administering a fixed dose combination (single dosage form) of therapeutically effective dose of ambrisentan or a pharmaceutically acceptable salt thereof to riociguat or a pharmaceutically acceptable salt thereof to a patient in need thereof. In one embodiment, the fixed dose combination comprises a 5 mg or 10 mg dose of ambrisentan a dose of riociguat or a pharmaceutically acceptable salt selected from the group consisting of 0.5 mg, 1.0 mg, 1.5 mg, 2.0 mg and 2.5 mg.

Also provided, in one aspect, is a method for treating or preventing a disease in a patient in need thereof comprising administering to the patient an effective amount of ambrisentan or a pharmaceutically acceptable salt thereof in combination with an effective amount of riociguat or a pharmaceutically acceptable salt thereof, wherein the disease is selected from the group consisting of hypertension, pulmonary hypertension, myocardial infarction, angina pectoris, acute kidney failure, renal insufficiency, cerebral vasospasms, cerebral ischemia, subarachnoid hemorrhages, asthma, atherosclerosis, intravascular coagulation, restenosis after angioplasty, hypertension caused by ischemia or intoxication, kidney failure caused by ischemia or intoxication, Raynaud's syndrome and asthmatic airway condition. In one embodiment, the weight ratio of ambrisentan or a pharmaceutically acceptable salt thereof to riociguat or a pharmaceutically acceptable salt thereof is in a range from about 1:1 to about 1:10 or from about 1:1 to 10:1.

Also provided, in one aspect, is a method for reducing an effective dose of ambrisentan or a pharmaceutically acceptable salt thereof, the method comprising administering to the patient an effective amount of riociguat or a pharmaceutically acceptable salt thereof, wherein the effectiveness of administration of the ambrisentan and the riociguat is at least about 25% greater than the sum of effectiveness of mono-administrations of the ambrisentan and the riociguat. In one embodiment, the weight ratio of ambrisentan or a pharmaceutically acceptable salt thereof to riociguat or a pharmaceutically acceptable salt thereof is in a range from about 1:1 to about 1:10 or from about 10:1 to 1:1. In another aspect, provided is a method for reducing an effective dose of riociguat or a pharmaceutically acceptable salt thereof comprising administering to the patient an effective amount of ambrisentan or a pharmaceutically acceptable salt thereof, wherein the effectiveness of administration of the ambrisentan and the riociguat is at least about 25% greater than the sum of effectiveness of mono-administrations of the ambrisentan and the riociguat. In one embodiment, the weight ratio of ambrisentan or a pharmaceutically acceptable salt thereof to riociguat or a pharmaceutically acceptable salt thereof is in a range from about 1:1 to about 1:10 or from about 10:1 to 1:1.

### Pulmonary Hypertension (PH)

The pulmonary hypertension condition treated by the method of the disclosure, can comprise any one or more of the conditions recognized according to the World Health Organization (WHO) or Venice (2003) classification (see, for example, Rubin (2004) Chest 126:7-10) or the most recent Dana Point classification (Simonneau (2009) JACC 54; 54:S43-S54):
Group 1: Pulmonary arterial hypertension (PAH)
   1.1 idiopathic PAH
   1.2 familial PAH
   1.3 PAH associated with:
   1.3.1 collagen vascular disease
   1.3.2 congenital systemic-to-pulmonary shunts (including Eisenmenger's syndrome)
   1.3.3 portal hypertension
   1.3.4 HIV infection
   1.3.5 drugs and toxins
   1.3.6 other (thyroid disorders, glycogen storage disease, Gaucher disease, hereditary hemorrhagic telangiectasia, hemoglobinopathies, myeloproliferative disorders, splenectomy)
   1.4 PAH associated with significant venous or capillary involvement
   1.4.1 pulmonary veno-occlusive disease (PVOD)
   1.4.2 pulmonary capillary hemangiomatosis (PCH)
   1.5 persistent pulmonary hypertension of the newborn
Group 2: Pulmonary hypertension with left heart disease
   2.1 left-sided atrial or ventricular heart disease
   2.2 left-sided valvular heart disease
Group 3: Pulmonary hypertension associated with lung diseases and/or hypoxemia
   3.1 chronic obstructive pulmonary disease (COPD)
   3.2 interstitial lung disease
   3.3 sleep-disordered breathing
   3.4 alveolar hypoventilation disorders
   3.5 chronic exposure to high altitude
   3.6 developmental abnormalities
Group 4: Pulmonary hypertension due to chronic thrombotic and/or embolic disease
   4.1 thromboembolic obstruction of proximal pulmonary arteries
   4.2 thromboembolic obstruction of distal pulmonary arteries
   4.3 non-thrombotic pulmonary embolism (tumor, parasites, foreign material)
Group 5: Miscellaneous (sarcoidosis, histiocytosis X, lymphangiomatosis, compression of pulmonary vessels (adenopathy, tumor, fibrosing mediastinitis)).

In one aspect, the pulmonary hypertension condition comprises PAH (WHO Group 1), for example idiopathic PAH, familial PAH or PAH associated with another disease or condition.

Pulmonary hypertension at baseline can be mild, moderate or severe, as measured for example by WHO functional class, which is a measure of disease severity in patients with pulmonary hypertension. The WHO functional classification is an adaptation of the New York Heart Association (NYHA) system and is routinely used to qualitatively assess activity tolerance, for example in monitoring disease progression and response to treatment (Rubin (2004) Chest 126:7-10). Four functional classes are recognized in the WHO system:

Class I: pulmonary hypertension without resulting limitation of physical activity; ordinary physical activity does not cause undue dyspnea or fatigue, chest pain or near syncope;

Class II: pulmonary hypertension resulting in slight limitation of physical activity; patient comfortable at rest; ordinary physical activity causes undue dyspnea or fatigue, chest pain or near syncope;

Class III: pulmonary hypertension resulting in marked limitation of physical activity; patient comfortable at rest; less than ordinary activity causes undue dyspnea or fatigue, chest pain or near syncope;

Class IV: pulmonary hypertension resulting in inability to carry out any physical activity without symptoms; patient manifests signs of right-heart failure; dyspnea and/or fatigue may be present even at rest; discomfort is increased by any physical activity.

In one aspect, the subject at baseline exhibits pulmonary hypertension (e.g., PAH) of at least WHO Class I, for example WHO Class I, II or Class III.

In another aspect, the subject at baseline exhibits mean PAP at rest of at least about 30 mmHg, for example at least about 35, at least about 40, at least about 45 or at least about 50 mmHg.

The methods of the present disclosure, when applied to a subject, can achieve one or more of the following objectives:

(a) adjustment of one or more hemodynamic parameters towards a more normal level, for example lowering mean PAP or PVR, raising cardiac output or index, or lowering PCWP or LVEDP, versus baseline;

(b) improvement of pulmonary function versus baseline, for example increasing exercise capacity or activity, illustratively as measured in a test of 6-minute walking distance (6 MWD) or measure of activity, or lowering Borg dyspnea index (BDI);

(c) improvement of one or more quality of life parameters versus baseline, for example an increase in score on at least one of the SF-36® health survey functional scales;

(d) general improvement versus baseline in the severity of the condition, for example by movement to a lower WHO functional class;

(e) improvement of clinical outcome following a period of treatment, versus expectation in absence of treatment (e.g., in a clinical trial setting, as measured by comparison with placebo), including improved prognosis, extending time to or lowering probability of clinical worsening, extending quality of life (e.g., delaying progression to a higher WHO functional class or slowing decline in one or more quality of life parameters such as SF-36® health survey parameters), and/or increasing longevity; and/or

(f) adjustment towards a more normal level of one or more molecular markers that can be predictive of clinical outcome (e.g., plasma concentrations of endothelin-1 (ET-1), cardiac troponin T (cTnT) or B-type natriuretic peptide (BNP)).

What constitutes an effective amount for treating PH, or in particular, PAH, can vary depending on the particular pulmonary hypertension condition to be treated, the severity of the condition, body weight and other parameters of the individual subject, and can be readily established without undue experimentation by the physician or clinician based on the disclosure herein.

Various clinical parameters and standards to measure the effectiveness of a PH therapy are described below and are known in the art as well. Accordingly, the effectiveness of a PH therapy, such as that of any combination formulation of the present disclosure, can be measured by these parameters or standards. Additionally, the relative effectiveness of a therapy, such as that of a combination of two agents, as compared to the effectiveness of mono-administrations of each agent, can be determined with these clinical parameters or standards, as well as in a non-clinical setting. Examples of such non-clinical settings include, without limitation, an in vitro assay or animal study. Non-limiting examples of in vitro assays are provided in Examples.

### A. Improvement on Clinical Parameters

In one aspect, the subject being treated experiences, during or following the treatment period, at least one of

(a) adjustment of one or more hemodynamic parameters indicative of the pulmonary hypertension condition towards a more normal level versus baseline;

(b) increase in exercise capacity versus baseline;

(c) lowering of BDI versus baseline;

(d) improvement of one or more quality of life parameters versus baseline; and/or

(e) movement to a lower WHO functional class.

Any suitable measure of exercise capacity can be used; a particularly suitable measure is obtained in a 6-minute walk test (6MWT), which measures how far the subject can walk in 6 minutes, i.e., the 6-minute walk distance (6MWD).

The Borg dyspnea index (BDI) is a numerical scale for assessing perceived dyspnea (breathing discomfort). It measures the degree of breathlessness after completion of the 6 minute walk test (6MWT), where a BDI of 0 indicates no breathlessness and 10 indicates maximum breathlessness.

In various aspects, an effective amount of a PH therapy adjusts one or more hemodynamic parameters indicative of the pulmonary hypertension condition towards a more normal level. In one such aspect, mean PAP is lowered, for example by at least about 3 mmHg, or at least about 5 mmHg, versus baseline. In another such aspect, PVR is lowered. In yet another such aspect, PCWP or LVEDP is raised.

In various aspects, an effective amount of a PH therapy improves pulmonary function versus baseline. Any measure of pulmonary function can be used; illustratively 6MWD is increased or BDI is lowered.

In one such aspect, 6MWD is increased from baseline by at least about 10 m, for example at least about 20 m or at least about 30 m. In many instances, the method of the present embodiment will be found effective to increase 6MWD by as much as 50 m or even more.

In another such aspect, BDI, illustratively as measured following a 6MWT, is lowered from baseline by at least about 0.5 index points. In many instances, the method of the present embodiment will be found effective to lower BDI by as much as 1 full index point or even more.

The SF-36® health survey provides a self-reporting, multi-item scale measuring eight health parameters: physical functioning, role limitations due to physical health problems, bodily pain, general health, vitality (energy and fatigue), social functioning, role limitations due to emotional problems, and mental health (psychological distress and psychological well-being). The survey also provides a physical component summary and a mental component summary.

In various aspects, an effective amount of a PH therapy can improve quality of life of the subject, illustratively as measured by one or more of the health parameters recorded in an SF-36® survey. For example, an improvement versus baseline is obtained in at least one of the SF-36 physical health related parameters (physical health, role-physical, bodily pain and/or general health) and/or in at least one of the SF-36 mental health related parameters (vitality, social functioning, role-emotional and/or mental health). Such an improvement can take the form of an increase of at least 1, for example at least 2 or at least 3 points, on the scale for any one or more parameters.

### B. Improvement of Prognosis

In another embodiment, the treatment method of the present disclosure can improve the prognosis for a subject having a pulmonary hypertension condition. The treatment of this embodiment can provide (a) a reduction in probability of a clinical worsening event during the treatment period, and/or (b) a reduction from baseline in serum brain natriuretic peptide (BNP) or NT pro-BNP or its N-terminal prohormone, NT-pro-BNP concentration, wherein, at baseline, time from first diagnosis of the condition in the subject is not greater than about 2 years.

Time from first diagnosis, in various aspects, can be, for example, not greater than about 1.5 years, not greater than about 1 year, not greater than about 0.75 year or not greater than about 0.5 year. In one aspect, administration of ambrisentan can begin substantially immediately, for example, within about one month or within about one week, upon diagnosis.

In this embodiment, the treatment period is long enough for the stated effect to be produced. Typically, the longer the treatment continues the greater and more lasting will be the benefits. Illustratively, the treatment period can be at least about one month, for example at least about 3 months, at least about 6 months or at least about 1 year. In some cases, administration can continue for substantially the remainder of the life of the subject.

Clinical worsening event (CWEs) include death, lung transplantation, hospitalization for the pulmonary hypertension condition, atrial septostomy, initiation of additional pulmonary hypertension therapy or an aggregate thereof. Therefore, the treatments of the present disclosure can be effective to provide a reduction of at least about 25%, for example at least about 50%, at least about 75% or at least about 80%, in probability of death, lung transplantation, hospitalization for pulmonary arterial hypertension, atrial septostomy and/or initiation of additional pulmonary hypertension therapy during the treatment period.

Time to clinical worsening of the pulmonary hypertension condition is defined as the time from initiation of an ambrisentan treatment regime to the first occurrence of a CWE.

In another particular aspect, the method is effective to provide a reduction from baseline of at least about 15%, for example at least about 25%, at least about 50% or at least about 75%, in BNP or NT-pro-BNP concentration.

The pulmonary hypertension condition according to this embodiment can comprise any one or more of the conditions in the WHO, Venice (2003) or Dana Point (2009) classifications described above. In one aspect, the condition comprises PAH (WHO Group 1), for example idiopathic PAH, familial PAH or PAH associated with another disease.

In various aspects of this embodiment, the subject at baseline exhibits PH (e.g., PAH) of WHO Class I-IV, for example Class I, Class II, Class III or Class IV as described above.

In a more particular embodiment, the subject at baseline has a resting PAP of at least about 25 mmHg, for example at least about 30 mmHg, at least about 35 mmHg or at least about 40 mmHg

In various aspects of this embodiment, the subject can experience, during or following the treatment period, at least one of:

(a) adjustment of one or more hemodynamic parameters indicative of improvement of the cardiopulmonary hypertension condition towards a more normal level versus baseline;

(b) improvement in cardiopulmonary function; illustratively an increase in exercise capacity or surrogate thereof (e.g., CPET measures such as VO₂ peak, VE/VCO₂, PETCO₂ and the like) or lowering of BDI versus baseline;

(c) improvement of one or more quality of life parameters versus baseline; and/or

(d) maintenance of or movement to a lower WHO functional class.

For example, in one aspect the subject can experience improvement in cardiopulmonary function versus baseline. Any measure of cardiopulmonary function can be used; illustratively 6MWD is increased or BDI is lowered.

In one such aspect, 6MWD is improved from baseline by at least about 10 m, for example, at least about 20 m or at least about 30 m. In many instances, the method of the present embodiment will be found effective to increase 6MWD by as much as 50 m or even more.

In another such aspect, BDI, illustratively as measured following a 6MWT, is lowered from baseline by at least about 0.5 point. In many instances, the method of the present embodiment will be found effective to lower BDI by as much as 1 full index point or even more.

In another aspect, the subject can experience improvement in quality of life, illustratively as measured by one or more of the health parameters recorded in an SF-360 survey. For example, an improvement versus baseline can be obtained in at least one of the SF-36 physical health related parameters (physical health, role-physical, bodily pain and/or general health) and/or in at least one of the SF-35 mental health related parameters (vitality, social functioning, role-emotional and/or mental health). Such an improvement can take the form of an increase of at least 1, for example at least 2 or at least 3 points, on the scale for any one or more parameters.

In another aspect, the subject can experience maintenance or improvement in WHO functional class.

### C. Prolongation of Life

In yet another embodiment, the treatment methods of the present disclosure can prolong the life of a subject having a pulmonary hypertension condition, from a time of initiation of treatment, by at least about 30 days. Variants and illustrative modalities of this method are as set forth above.

### D. Extending Time to Clinical Worsening

Still in another embodiment, the present methods can extend time to clinical worsening in a subject having a pulmonary hypertension condition, and decrease the probability of a clinical worsening event by at least about 25%. Variants and illustrative modalities of this method are as set forth above.

### E. Other Treatment Objectives

In any of the methods described hereinabove, the subject can be male or female. For example, the combined drugs can be administered to a female subject according to any of the above methods, including the indicated variants and illustrative modalities thereof. Alternatively, ambrisentan can be administered to a male subject, for example a reproductively active male subject, according to any of the above methods, including the indicated variants and illustrative modalities thereof.

In another embodiment, the methods provided herein are useful for treating a pulmonary hypertension condition in a reproductively active male subject, wherein fertility of the subject is not substantially compromised. “Not substantially compromised” in the present context means that spermatogenesis is not substantially reduced by the treatment and that no hormonal changes are induced that are indicative of or associated with reduced spermatogenesis. Male fertility can be assessed directly, for example, by sperm counts from semen samples, or indirectly by changes in hormones such as follicle stimulating hormone (FSH), luteinizing hormone (LH), inhibin B and testosterone.

In one embodiment, a method is provided for treating PAH in a subject, wherein the PAH is associated with one or more of (a) a congenital heart defect, (b) portal hypertension, (c) use of a drug or toxin other than an anorexigen, (d) thyroid disorder, (e) glycogen storage disease, (f) Gaucher disease, (g) hereditary hemorrhagic telangiectasia, (h) hemoglobinopathy, (i) myeloproliferative disorder, (j) splenectomy, (k) pulmonary veno-occlusive disease and/or (l) pulmonary capillary hemangiomatosis. Variants and illustrative modalities of this method are as set forth hereinabove.

Further, in another embodiment, a method is provided for treating a pulmonary hypertension condition classified in WHO Groups 2-5 in a subject. Variants and illustrative modalities of this method are as set forth hereinabove. In one aspect, the condition comprises left-sided atrial or ventricular heart disease and/or left-sided valvular heart disease. In another aspect, the condition is associated with one or more of chronic obstructive pulmonary disease (COPD), interstitial lung disease (ILD), sleep-disordered breathing, an alveolar hypoventilation disorder, chronic exposure to high altitude, a developmental abnormality, thromboembolic obstruction of proximal and/or distal pulmonary arteries, a non-thrombotic pulmonary embolism, sarcoidosis, histiocytosis X, lymphangiomatosis, and/or compression of pulmonary vessels.

### Other Uses of the Combinations

Increased or abnormal release of endothelin causes persistent vasoconstriction in the peripheral, renal and cerebral blood vessels, which may lead to illnesses. It has been reported in the literature that elevated plasma levels of endothelin were found in patients with hypertension, acute myocardial infarct, pulmonary hypertension, Raynaud's syndrome, atherosclerosis and in the airways of asthmatics (see, e.g., U.S. Pat. No. 7,601,730). Accordingly, substances which inhibit the binding of endothelin to the receptor ought also to antagonize the various abovementioned physiological effects of endothelin and therefore be valuable drugs, such as ambrisentan and riociguat. Likewise, the combination of such drugs can also be effective in treating such diseases and conditions.

Thus, in one aspect, the present disclosure provides a method for treating or preventing a disease in a patient in need thereof comprising administering to the patient therapeutic amounts of a selective type-A ERA and a stimulator of soluble guanylate cyclase, wherein the disease is selected from the group consisting of hypertension, pulmonary hypertension, myocardial infarction, angina pectoris, acute kidney failure, renal insufficiency, cerebral vasospasms, cerebral ischemia, subarachnoid hemorrhages, asthma, atherosclerosis, intravascular coagulation, restenosis after angioplasty, hypertension caused by ischemia or intoxication, kidney failure caused by ischemia or intoxication, Raynaud's syndrome and asthmatic airway condition.

Also provided, is a method for inhibiting vasoconstriction in a patient in need thereof comprising administering to the patient therapeutic amounts of ambrisentan and riociguat or pharmaceutically acceptable salt thereof. In one aspect, the vasoconstriction is endothelin-induced.

It is also contemplated that by combining ambrisentan and riociguat any undesired side effects may be reduced. For example, administration of ambrisentan to a patient already receiving riociguat therapy reduces the side effects of riociguat. The co-action effect of combined administration will allow for a reduction in amount of riociguat necessary to achieve a therapeutic effect, thereby resulting in a reduced incidence of undesirable side effects. As such, in one embodiment, the disclosure is directed to a method for reducing the undesirable side effects of riociguat or a pharmaceutically acceptable salt thereof comprising administering a therapeutic amount of ambrisentan or a pharmaceutically acceptable salt thereof.

As discussed above, by administration of ambrisentan, the therapeutically effective amount of riociguat is reduced. As such, the disclosure, in one embodiment, is directed to a method for reducing the therapeutically effective dose of riociguat or a pharmaceutically acceptable salt thereof comprising administering to a patient a therapeutic amount of ambrisentan or a pharmaceutically acceptable salt thereof.

### Dosing

For all of the methods just described, at least one of either ambrisentan or a pharmaceutically acceptable salt thereof or riociguat or a pharmaceutically acceptable salt thereof is administered in a less than standard therapeutic dose which becomes therapeutically effective as a consequence of its administration with the other drug. However, it is also contemplated that riociguat and ambrisentan may also both be administered in an effective amount. In some embodiments, the riociguat is administered in an effective dose and ambrisentan is administered in a standard therapeutically effective dose. In other embodiment, ambrisentan is administered in a less than standard therapeutic dose and riociguat is administered in a standard therapeutically effective dose. In still other embodiments, both ambrisentan and riociguat are administered in less than standard therapeutic doses. The expression “therapeutically effective amounts of riociguat and ambrisentan or a pharmaceutically acceptable salt thereof” is intended to encompass all possible combinations of standard and less than standard therapeutic doses of ambrisentan or its therapeutically acceptable salt and riociguat or its therapeutically acceptable salt.

In some embodiments, riociguat or a pharmaceutically acceptable salt thereof and ambrisentan or a pharmaceutically acceptable salt thereof are administered together as in a combined or fixed dose, separately but simultaneously, separately or sequentially within a time period effective to provide enhanced efficacy. The time period ranges from a few minutes to several hours. The time period is, for example, 5 minutes, 1.0 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, or 8 hours.

Ambrisentan and riociguat may be given to the patient in either single or multiple doses by any of the accepted modes of administration of agents having similar utilities, for example as described in those patents and patent applications incorporated by reference, including buccal, by intra-arterial injection, intravenously, intraperitoneally, parenterally, intramuscularly, subcutaneously, orally, or via an impregnated or coated device such as a stent, for example, or an artery-inserted cylindrical polymer. In one embodiment, ambrisentan or a pharmaceutically acceptable salt thereof to riociguat or a pharmaceutically acceptable salt thereof are administered intravenously.

In one embodiment, ambrisentan or a pharmaceutically acceptable salt thereof to riociguat or a pharmaceutically acceptable salt thereof are administered orally. Riociguat or a pharmaceutically acceptable salt thereof and ambrisentan or a pharmaceutically acceptable salt thereof may also be administered as a combined dosage unit, such as, for example, in a tablet.

As mentioned above, riociguat or a pharmaceutically acceptable salt thereof and ambrisentan or a pharmaceutically acceptable salt thereof may be administered in a therapeutic amount or an effective amount. Therefore, in some embodiments, the amount of ambrisentan or a pharmaceutically acceptable salt thereof administered is from about 0.1 mg to about 100 mg, or from about 0.1 mg to about 20 mg, or from about 0.1 mg to about 10 mg, or from about 0.25 mg to about 10 mg, or from about 0.25 mg to about 5 mg, daily. In certain embodiments, the amount of ambrisentan or a pharmaceutically acceptable salt thereof administered is about 0.1 mg, or about 0.25 mg, or about 0.5 mg, or about 1 mg, or about 1.5 mg, or about 2 mg, or about 3 mg, or about 4 mg, or about 5 mg, or about 7.5 mg, or about 10 mg.

Further, the amount of riociguat or a pharmaceutically acceptable salt thereof administered is from about 0.1 to about 100 mg, or from about 0.1 mg to about 20 mg, or from about 0.1 mg to about 10 mg, or from about 0.25 mg to about 50 mg, or from about 0.25 mg to about 10 mg, or from about 0.25 mg to about 7.5 mg, or from about 0.5 mg to about 7.5 mg, daily. These daily doses may be administered to the patient either once, twice or three times per day. In certain embodiments, the amount of riociguat or a pharmaceutically acceptable salt thereof administered is about 0.1 mg, or about 0.25 mg, or about 0.5 mg, or about 1 mg, or about 1.5 mg, or about 2 mg, or about 3 mg, or about 4 mg, or about 5 mg, or about 6 mg, or about 7.5 mg, or about 10 mg.

In one embodiment, whether ambrisentan and riociguat are administered together or separately, the ratio of the amount of ambrisentan or a pharmaceutically acceptable salt thereof and the amount of riociguat or a pharmaceutically acceptable salt thereof can be in a range from about 1:1 to about 1:10, or alternatively from about 1:2 to about 1:8, or alternatively from about 1:2 to about 1:5, or alternatively from about 1:2.5 to about 1:3.5 or in a particular aspect, is about 1:3.

Additionally, ambrisentan or a pharmaceutically acceptable salt thereof is administered as a sustained release formulation and/or riociguat or a pharmaceutically acceptable salt thereof is administered as an immediate release or sustained release formulation. This is more thoroughly discussed in the next section.

In one embodiment then, the patient under treatment is already taking a maintenance dose of riociguat ranging from about 2.5 mg to about 7.5 mg with a typical dose once daily. To this dosing regimen is then added ambrisentan at from about 5 mg to about 10 mg. By administering such therapeutic doses of ambrisentan the amount of riociguat may then be decreased to from about 2.5-7.5 to about 1-5 mg or about 0.1-2.5 mg daily thereby greatly reducing the incidence of adverse events. Likewise, by administering such therapeutic doses of riociguat the amount of ambrisentan can then be decreased to from about 5-10 to about 3-8 mg or about 2-5 mg daily thereby greatly reducing the incidence of adverse events.

### Active Ingredients and Compositions

Pharmaceutical Formulations

This disclosure also provides pharmaceutical formulations. One aspect provides a pharmaceutical formulation comprising an effective amount of ambrisentan or a pharmaceutically acceptable salt thereof in combination with an effective amount of riociguat or a pharmaceutically acceptable salt thereof. In one embodiment, the weight ratio of ambrisentan or a pharmaceutically acceptable salt thereof to riociguat or a pharmaceutically acceptable salt thereof is in a range from about 1:1 to about 1:10 or from about 1:1 to 10:1. In another embodiment, the effectiveness of combination of the ambrisentan or a pharmaceutically acceptable salt thereof to riociguat or a pharmaceutically acceptable salt thereof is at least about 25% greater than the sum of effectiveness of mono-administrations of ambrisentan or a pharmaceutically acceptable salt thereof and the riociguat or a pharmaceutically acceptable salt thereof.

As mentioned above, a combination of riociguat and ambrisentan may be formulated separately. The separate dosage forms containing each active ingredient can be administered sequentially or at similar times (i.e., either together or one after the other). In another embodiment, riociguat and ambrisentan is co-formulated into a combined dosage unit. Accordingly, in one embodiment, the disclosure is directed to pharmaceutical formulations comprising a therapeutic amount of riociguat or a pharmaceutically acceptable salt thereof, a therapeutic amount of ambrisentan or a pharmaceutically acceptable salt thereof, and a pharmaceutically acceptable carrier.

In another embodiment, the formulation comprises an effective amount of ambrisentan or a pharmaceutically acceptable salt thereof and/or riociguat or a pharmaceutically acceptable salt thereof. In certain embodiments, the formulations are formulated for either intravenous or oral administration. In still other embodiment, the two active ingredients are co-formulated into a combined dosage unit. In still yet other embodiments, the two active ingredients are formulated separately for co-therapy administration.

Co-Formulations

In certain embodiments of the present disclosure, the ambrisentan and riociguat are co-formulated into a combined dosage unit or unitary dosage form suitable for oral administration. In certain embodiments, the ambrisentan is formulated as a sustained release formulation. In certain embodiments, the riociguat is formulated for immediate release or sustained release.

In one embodiment, the formulation is in tablet form or capsule form. In another embodiment, the tablet or capsule comprises from about 0.25 mg to about of 100 mg of riociguat or a pharmaceutically acceptable salt thereof. In another embodiment, the tablet or capsule comprises from about 0.25 mg to about 50 mg of riociguat or a pharmaceutically acceptable salt thereof. In yet another embodiment, the tablet or capsule comprises from about 0.25 mg to about 10 mg of riociguat or a pharmaceutically acceptable salt thereof. In yet another embodiment, the tablet or capsule comprises from about 0.5 mg to about 7.5 mg of riociguat or a pharmaceutically acceptable salt thereof. In yet another embodiment, the tablet or capsule comprises from about 0.5 mg to about 5 mg of riociguat or a pharmaceutically acceptable salt thereof. In yet another embodiment, the tablet or capsule comprises from about 0.5 mg to about 2.5 mg of riociguat or a pharmaceutically acceptable salt thereof.

In one embodiment, the tablet or capsule comprises from about 0.1 mg to about 35 mg of ambrisentan or a pharmaceutically acceptable salt thereof. In another embodiment, the tablet or capsule comprises from about 0.1 mg to about 20 mg of ambrisentan or a pharmaceutically acceptable salt thereof. In yet another embodiment, the tablet or capsule comprises from about 0.25 mg to about 10 mg of ambrisentan or a pharmaceutically acceptable salt thereof. In yet another embodiment, the tablet or capsule comprises from about 0.25 mg to about 5 mg of ambrisentan or a pharmaceutically acceptable salt thereof. In yet another embodiment, the tablet of capsule comprises about 5 mg or about 10 mg ambrisentan. Thus, one embodiment of the disclosure is a pharmaceutical composition comprising 5 mg or 10 mg of ambrisentan or a pharmaceutically acceptable salt thereof, in combination with 0.1 mg, 0.5 mg, 1 mg, 1.5 mg, 2.0 mg or 2.5 mg of riociguat or a pharmaceutically acceptable salt and one or more excipients.

In one embodiment, the ratio of the amount of ambrisentan or a pharmaceutically acceptable salt thereof and the amount of riociguat or a pharmaceutically acceptable salt thereof, in the formulation, can be from about 1:1 to about 1:10, or alternatively from about 1:2 to about 1:8, or alternatively from about 1:2 to about 1:5, or alternatively from about 1:2.5 to about 1:3.5 or in a particular aspect, is about 1:3.

In one embodiment, the ratio of the amount of ambrisentan or a pharmaceutically acceptable salt thereof and the amount of riociguat or a pharmaceutically acceptable salt thereof, in the formulation, can be from about 1:1 to about 10:1, or alternatively from about 1:1 to about 8:1, or alternatively from about 1:1 to about 5:1, or alternatively from about 1:1 to about 3.5:1 or is about 2:1.

In one such embodiment, the ambrisentan composition is placed in a portion of the tablet which is separate from, but in contact with, the portion of the tablet containing the riociguat composition. It will be understood that the unitary dosage form may comprise simply compressing the ambrisentan composition and the riociguat composition into a multilayer tablet or conventionally processed into other conventional unitary dosage forms such as a capsules. The multilayer tablets and capsules suitable for use in the present disclosure can be fabricated using methods known in the art using standard machinery.

The tablets may comprise two layers, i.e. a first layer which comprises the riociguat and is formulated for immediate release or sustained release, and a second layer which comprises the ambrisentan and is formulated for sustained release. Alternatively, the multilayer tablet may comprise an inner layer and an outer layer, where the inner layer comprises the sustained release ambrisentan formulation and where the outer layer comprises the immediate release or sustained release riociguat layer. In another embodiment, the ambrisentan and riociguat are co-formulated into a capsule, where the capsule allows for the immediate release or sustained release of riociguat and the sustained release of ambrisentan. For example, the capsule may contain granules of both riociguat and ambrisentan, where the granules have been formulated such that the riociguat is available for immediate release or sustained release and the Ambrisentan is formulated for sustained release. Alternatively, the capsule may contain a liquid immediate release or sustained release formulation of riociguat and a solid sustained release formulation of ambrisentan. However, such embodiments are exemplary and are not intended to limit the formulations of the present disclosure.

A multilayer tablet can be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active agent or dispersing agent. Molded tablets may be made by molding in a suitable machine a mixture of the powdered active ingredient moistened with an inert liquid diluent. The tablets may optionally be coated or scored.

The tablets may contain one or more agents including sweetening agents, flavoring agents, coloring agents and preserving agents, in order to provide a palatable preparation. Tablets containing the active ingredients in admixture with non-toxic pharmaceutically acceptable excipients which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Other Formulations

Formulations also contemplated by the present disclosure may also be for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present disclosure. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. The same formulations are contemplated for separate administration of ambrisentan and riociguat.

Sterile injectable solutions are prepared by incorporating the component in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The ideal forms of the apparatus for administration of the novel combinations for pulmonary hypertension and other methods of the disclosure consist therefore of (1) either a syringe comprising 2 compartments containing the 2 active substances ready for use or (2) a kit containing two syringes ready for use.

In making a pharmaceutical composition including ambrisentan and riociguat, the active ingredients are usually diluted by an excipient or carrier and/or enclosed within such a carrier that can be in the form of a capsule, sachet, paper or other container. When the excipient serves as a diluent, in can be a solid, semi-solid, or liquid material (as above), which acts as a vehicle, carrier or medium for the active ingredient. Thus, the compositions can be in the foam of tablets, pills, powders, lozenges, sachets, cachets, elixirs, suspensions, emulsions, solutions, syrups, aerosols (as a solid or in a liquid medium), ointments containing, for example, up to 10% by weight of the active compounds, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the disclosure can be formulated so as to provide quick, sustained or delayed release of the active ingredient after administration to the patient by employing procedures known in the art. As discussed above, given the reduced bioavailability of ambrisentan, sustained release formulations are generally preferred. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345.

The compositions are preferably formulated in a unit dosage form. The term “unit dosage forms” or “combined dosage unit” refers to physically discrete units suitable as unitary dosages for human subjects and other mammals, each unit containing a predetermined quantity of the active materials calculated to produce the desired therapeutic effect, in association with a suitable pharmaceutical excipient (e.g., a tablet, capsule, ampoule). The active agents of the disclosure are effective over a wide dosage range and are generally administered in a pharmaceutically effective amount. It will be understood, however, that the amount of each active agent actually administered will be determined by a physician, in the light of the relevant circumstances, including the condition to be treated, the chosen route of administration, the actual compounds administered and their relative activity, the age, weight, and response of the individual patient, the severity of the patient's symptoms, and the like.

For preparing solid compositions such as tablets, the principal active ingredients are mixed with a pharmaceutical excipient to form a solid preformulation composition containing a homogeneous mixture of a compound of the present disclosure. When referring to these preformulation compositions as homogeneous, it is meant that the active ingredients are dispersed evenly throughout the composition so that the composition may be readily subdivided into equally effective unit dosage forms such as tablets, pills and capsules.

The tablets or pills of the present disclosure may be coated or otherwise compounded to provide a dosage form affording the advantage of prolonged action, or to protect from the acid conditions of the stomach. For example, the tablet or pill can comprise an inner dosage and an outer dosage element, the latter being in the form of an envelope over the former. Ambrisentan and the co-administered agent(s) can be separated by an enteric layer that serves to resist disintegration in the stomach and permit the inner element to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Additional embodiments of the disclosure include kits comprising a therapeutic amount of ambrisentan or a pharmaceutically acceptable salt thereof and a therapeutic amount of riociguat or a pharmaceutically acceptable salt thereof.

The following example is included to demonstrate preferred embodiments of the disclosure. It should be appreciated by those of skill in the art that the techniques disclosed in the example which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

In addition, abbreviations as used herein have respective meanings as follows:

| | |
|---|---|
| nM | nanomolar |
| pM | picomolar |
| SEM | standard error of the mean |
| cGMP | cyclic guanosine monophosphate |
| pmol | picomole |
| g | gram |
| MLCK | myosin light chain kinase |
| MLC2 | myosin light chain 2 |
| PKG | protein kinase G |
| CPET | cardiopulmonary exercise testing |
| VO₂ | oxygen uptake |
| PETCO₂ | amount of CO₂ in the exhaled air |
| VE/VCO₂ | ventillatory efficiency |
| BDI | Borg dyspnea index |
| 6MWD | six minute walking distance |
| PAP | pulmonary artery pressure |

### EXAMPLES

### Example 1

### Ambrisentan and Riociguat Relax Endothelin-Induced Contraction of Rat Pulmonary Arteries while Non-Selective ERA and Stimulator of Soluble Guanylate Cyclase Lack Such Co-Action

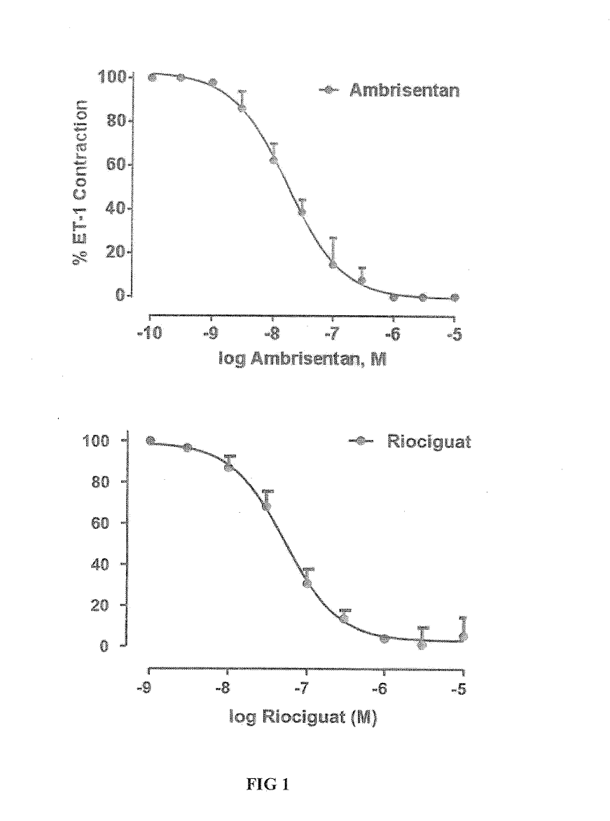

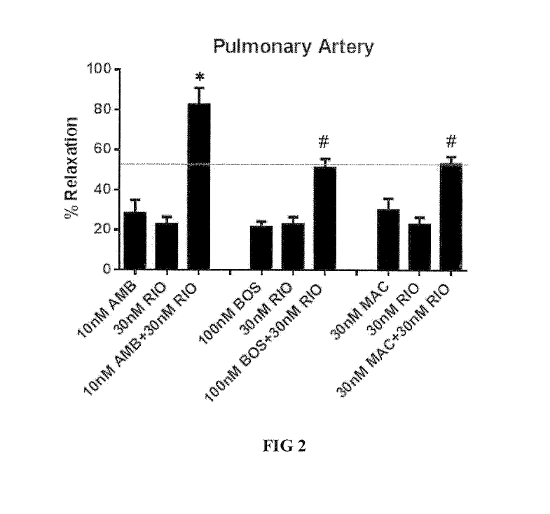

This example examines the pharmacological effects of the combination of ambrisentan and riociguat in comparison with either of them alone, with respect to their capability to relax isolated rat pulmonary arteries. The selective type-A endothelin receptor antagonist, ambrisentan (Letairis®), and the stimulator of soluble guanylate cyclase, riociguat (Adempas®), are currently used to treat pulmonary arterial hypertension. 10 nM ambrisentan and 30 nM riociguat were selected for combination study based on the concentration-dependent effects of ambrisentan and riociguat on ET-1-induced contraction of rat pulmonary arteries (FIG. 1) 10 nM ambrisentan and 30 nM riociguat alone relaxed 8 nM endothelin-1 (ET-1)-constricted intrapulmonary arteries isolated from rats by 29±6% (mean±SEM, n=6) and 23±3% (n=18), respectively, whereas both drugs in combination relaxed the intrapulmonary arterial rings by 83.0±8.0% (n=6, p<0.05 versus ambrisentan and riociguat alone or the calculated sum, that is, 54%, of individual inhibitions of each drug) (FIG. 2).

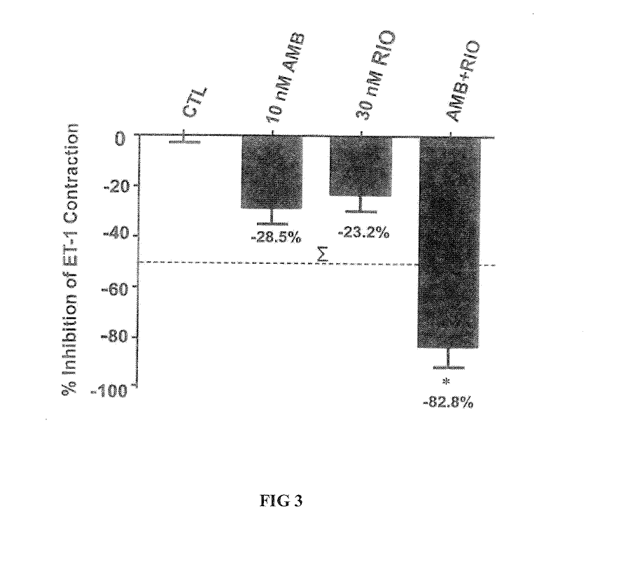

Another run of 10 nM ambrisentan and 30 nM riociguat alone relaxed 8 nM endothelin-1 (ET-1)-constricted intrapulmonary arteries isolated from rats by 28.5±6.4% (mean±SEM, n=6) and 23.2±6.5% (n=6), respectively, whereas both drugs in combination relaxed the intrapulmonary arterial rings by 82.8±8.0% (n=6, p<0.05 versus ambrisentan and riociguat alone or the calculated sum, that is, 51.8±7.6%, of individual inhibitions of each drug) (FIG. 3).

In contrast, the combination of 30 nM riociguat with 100 nM bosentan or 30 nM macitentan, two nonselective type-A & B endothelin receptor antagonists, relaxed the ET-1-dependent contraction of the intrapulmonary arterial rings by 51±4% or 53±3% (n=8, p<0.05 versus bosentan, macitentan and riociguat alone but no significant difference versus the calculated sum of individual inhibitions of each drug). 100 nM bosentan and 30 nM macitentan alone produced a vasorelaxant effect similar to 10 nM ambrisentan and relaxed the isolated intrapulmonary arterial rings contracted with 8 nM ET-1 by 23±3% (n=9) and 30±6% (n=12), respectively.

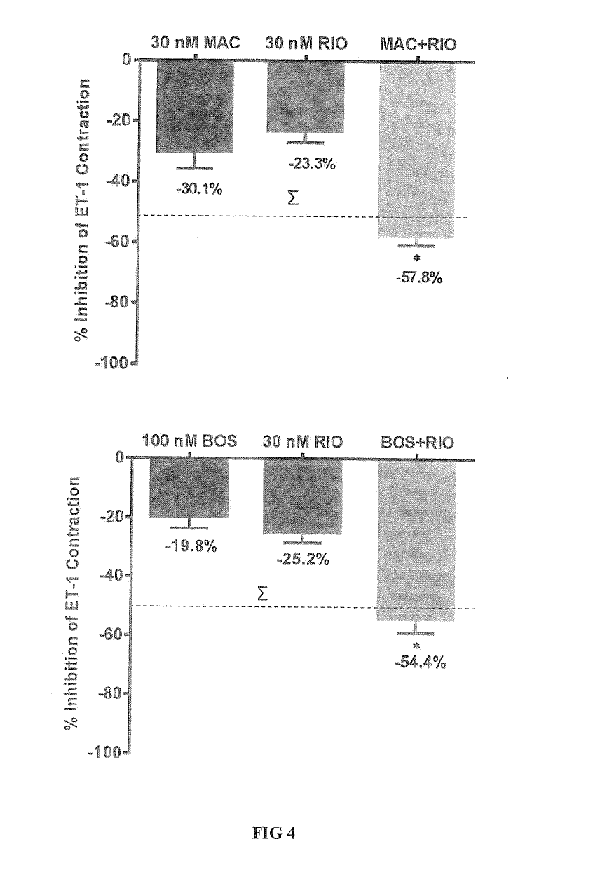

Another run of the combination of 30 nM riociguat with 100 nM bosentan or 30 nM macitentan, relaxed the ET-1-dependent contraction of the intrapulmonary arterial rings by 54.4% or 57.8% (n=6, p<0.05 versus bosentan, macitentan and riociguat alone but no significant difference versus the calculated sum of individual inhibitions of each drug). 100 nM bosentan and 30 nM macitentan alone produced a vasorelaxant effect similar to 10 nM ambrisentan and relaxed the isolated intrapulmonary arterial rings contracted with 8 nM ET-1 by 19.8% (n=6) and 30.1% (n=6), respectively (FIG. 4).

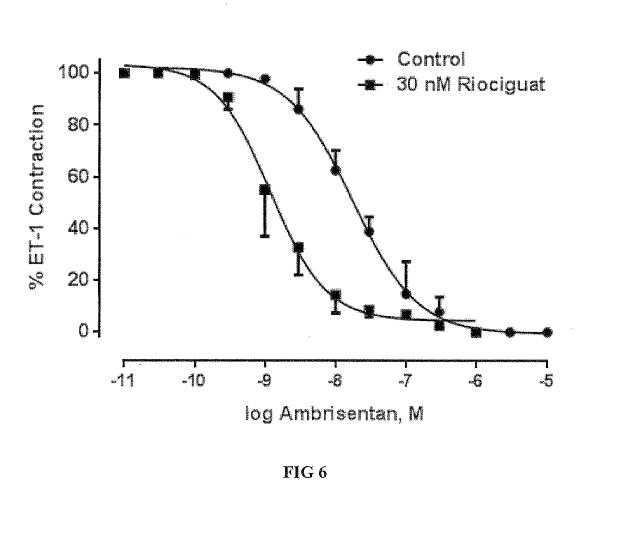

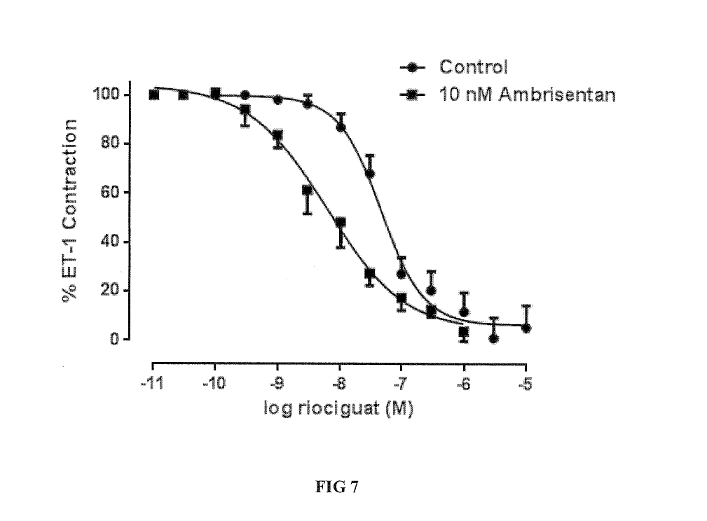

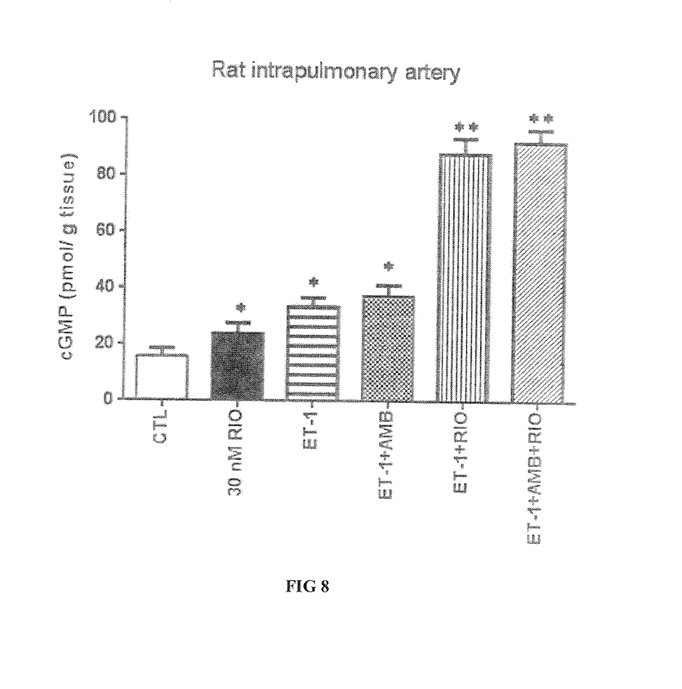

Riociguat increased the potency of ambrisentan and ambrisentan increased the potency of riociguat to relax ET-1 constricted rat pulmonary arteries. (FIG. 6 and FIG. 7). Furthermore, ET-1 and riociguat significantly increased cGMP synthesis, and riociguat potentiated ET-1-stimulated cGMP production in isolated rat intrapulmonary arteries. (FIG. 8). The contents of cGMP in isolated intrapulmonary arteries treated with vehicle, 10 nM ET-1, 30 nM riociguat or their combination were 17±2 pmol/g tissue (n=18), 37±4 pmol/g tissue (n=12, p<0.05 versus vehicle control), 34±5 pmol/g tissue (n=12, p<0.05 versus vehicle control) or 96±7 pmol/g tissue (p<0.01 versus ET-1 or riociguat). Bosentan and L-NAME (CAS 51298-62-5), but not ambrisentan, significantly blocked ET-1 and its combination with riociguat to stimulate cGMP synthesis in isolated intrapulmonary arteries. In the presence of 100 nM bosentan, and 8 nM ET-1 plus 30 nM riociguat-stimulated cGMP synthesis were reduced to 21±3 pmol/g tissue (n=12, p<0.05 versus ET-1) and 37±3 pmol/g tissue (n=6, p<0.05 versus ET-1+riociguat), respectively.

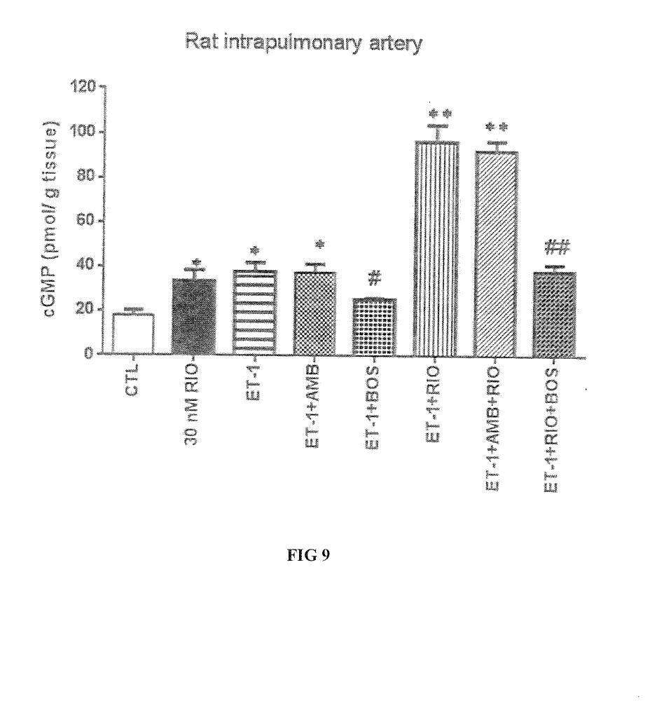

Another run in the presence of 100 nM bosentan, and 8 nM ET-1 plus 30 nM riociguat-stimulated cGMP synthesis were reduced to 36 pmol/g tissue (n=10, p<0.05 versus ET-1 or ET-1+ambrisentan) and 97 pmol/g tissue (n=10, p<0.01 versus ET-1+riociguat or ET-1+ambrisentan+riociguat), respectively (FIG. 9).

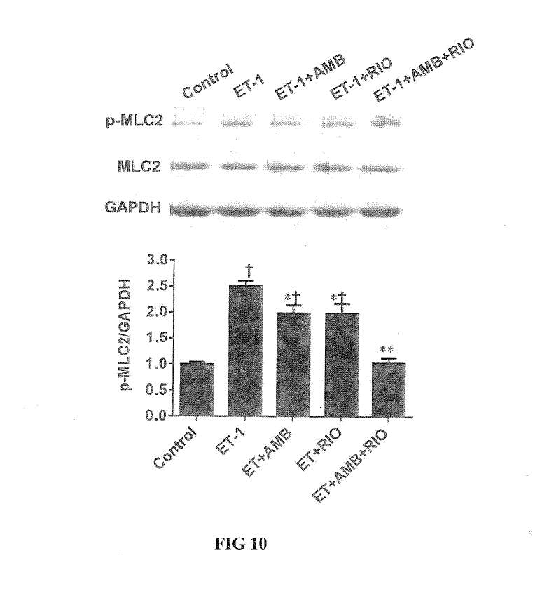

Further, ambrisentan and riociguat attenuated ET-1 stimulated phosphorylation of MLC2 by MLCK in rat pulmonary arteries (FIG. 10).

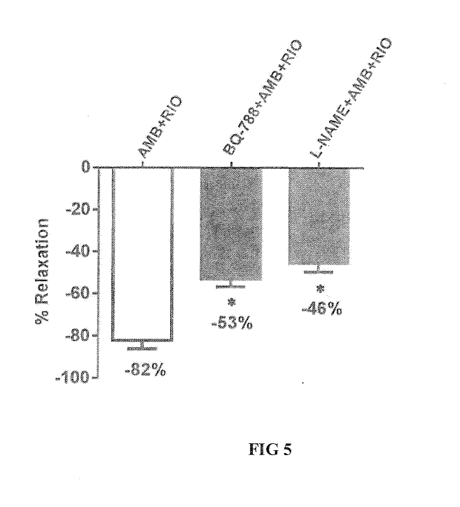

Taken together, these data suggest that ambrisentan and riociguat act to inhibit endothelin-induced contraction of pulmonary arteries and the type-B endothelin receptor-mediated nitric oxide production may contribute to their effect on pulmonary vasorelaxation. Endothelial ET_{B} receptor contributes to the effect of ambrisentan and riociguat on pulmonary vasorelaxation (FIG. 5).

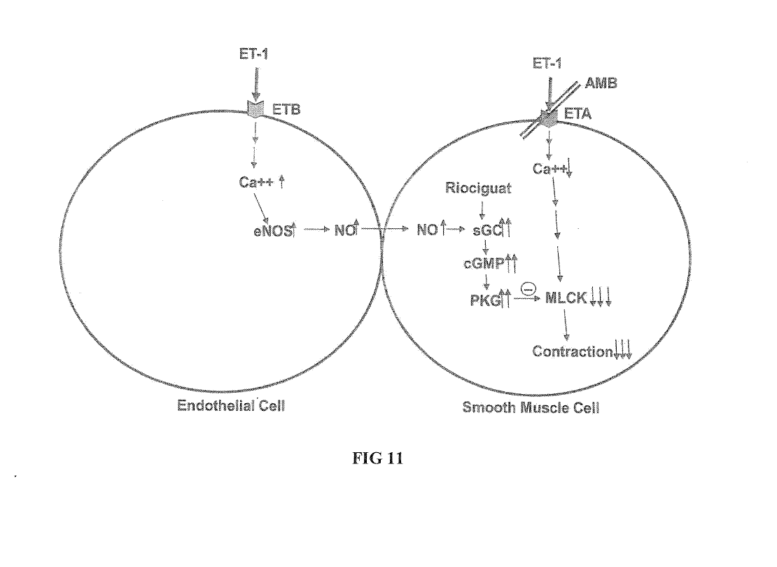

Ambrisentan selectively blocks the ETA receptors and thereby reduces ET-1 stimulated MLCK activity and contraction. Riociguat stimulates sGC activity and synergizes with NO to stimulate cGMP synthesis and thereby increases inhibition of MLCK by PKG (FIG. 11).

What is claimed is:

1. A method of treating pulmonary hypertension in a patient in need thereof, said method comprising administering to the patient an effective amount of ambrisentan or a pharmaceutically acceptable salt thereof in combination with an effective amount of riociguat or a pharmaceutically acceptable salt thereof.

2. A method of inhibiting endothelin-induced vasoconstriction in a patient comprising administering to the patient an effective amount of ambrisentan or a pharmaceutically acceptable salt thereof in combination with an effective amount of riociguat or a pharmaceutically acceptable salt thereof.

3. A method for treating a disease in a patient comprising administering to the patient an effective amount of ambrisentan or a pharmaceutically acceptable salt thereof in combination with an effective amount of riociguat or a pharmaceutically acceptable salt thereof, wherein the disease is selected from the group consisting of hypertension, pulmonary hypertension, pulmonary arterial hypertension, myocardial infarction, angina pectoris, acute kidney failure, renal insufficiency, cerebral vasospasms, cerebral ischemia, subarachnoid hemorrhages, asthma, atherosclerosis, intravascular coagulation, restenosis after angioplasty, hypertension caused by ischemia or intoxication, kidney failure caused by ischemia or intoxication, Raynaud's syndrome and asthmatic airway condition.

4. A method for reducing an effective dose of ambrisentan or a pharmaceutically acceptable salt thereof, the method comprising administering to the patient an effective amount of riociguat or a pharmaceutically acceptable salt thereof, wherein the effectiveness of administration of ambrisentan or a pharmaceutically acceptable salt thereof to riociguat or a pharmaceutically acceptable salt thereof is at least about 25% greater than the sum of effectiveness of mono-administrations of ambrisentan or a pharmaceutically acceptable salt thereof to riociguat or a pharmaceutically acceptable salt thereof.

5. A method for reducing an effective dose of riociguat or a pharmaceutically acceptable salt thereof comprising administering to the patient an effective amount of ambrisentan or a pharmaceutically acceptable salt thereof, wherein the effectiveness of administration of ambrisentan or a pharmaceutically acceptable salt thereof to riociguat or a pharmaceutically acceptable salt thereof is at least about 25% greater than the sum of effectiveness of mono-administrations of ambrisentan or a pharmaceutically acceptable salt thereof to riociguat or a pharmaceutically acceptable salt thereof.

6. The method of claim 1, wherein the weight ratio of ambrisentan or a pharmaceutically acceptable salt thereof to riociguat or a pharmaceutically acceptable salt thereof is in a range from about 1:1 to about 1:10.

7. The method of claim 1, wherein the weight ratio of ambrisentan or a pharmaceutically acceptable salt thereof to riociguat or a pharmaceutically acceptable salt thereof is in a range from about 1:2 to about 1:8.

8. The method of claim 1, wherein the weight ratio of ambrisentan or a pharmaceutically acceptable salt thereof to riociguat or a pharmaceutically acceptable salt thereof is in a range from about 1:1 to about 1:5.

9. The method of claim 1, wherein the weight ratio of ambrisentan or a pharmaceutically acceptable salt thereof to riociguat or a pharmaceutically acceptable salt thereof is about 1:3.

10. The method of claim 1, wherein the weight ratio of ambrisentan or a pharmaceutically acceptable salt thereof to riociguat or a pharmaceutically acceptable salt thereof is in a range from about 1:1 to about 100:1.

11. The method of claim 1, wherein the weight ratio of ambrisentan or a pharmaceutically acceptable salt thereof to riociguat or a pharmaceutically acceptable salt thereof is in a range from about 1:1 to about 10:1.

12. The method of claim 1, wherein the weight ratio of ambrisentan or a pharmaceutically acceptable salt thereof to riociguat or a pharmaceutically acceptable salt thereof is about 8:1.

13. The method of claim 1, wherein the weight ratio of ambrisentan or a pharmaceutically acceptable salt thereof to riociguat or a pharmaceutically acceptable salt thereof is about 5:1.

14. The method of claim 1, wherein ambrisentan or a pharmaceutically acceptable salt thereof to riociguat or a pharmaceutically acceptable salt thereof are formulated separately.

15. The method of claim 1, wherein ambrisentan or a pharmaceutically acceptable salt thereof to riociguat or a pharmaceutically acceptable salt thereof are formulated in a fixed dose formulation.

16. The method of claim 1, wherein ambrisentan or a pharmaceutically acceptable salt thereof to riociguat or a pharmaceutically acceptable salt thereof are formulated for parenteral administration.

17. The method of claim 1, wherein ambrisentan or a pharmaceutically acceptable salt thereof to riociguat or a pharmaceutically acceptable salt thereof are formulated for oral administration.

18. The method of claim 1, wherein ambrisentan or a pharmaceutically acceptable salt thereof to riociguat or a pharmaceutically acceptable salt thereof are formulated in tablet form or capsule form.

19. The method of claim 1, wherein the amount of ambrisentan or a pharmaceutically acceptable salt thereof is from about 0.1 mg to about 10 mg daily.

20. The method of claim 1, wherein the amount of ambrisentan or a pharmaceutically acceptable salt thereof is from about 0.2 mg to about 5 mg daily.

21. The method of claim 1, wherein the amount of riociguat or a pharmaceutically acceptable salt thereof administered is from about 0.1 mg to about 10 mg daily.

22. The method of claim 1, wherein the amount of riociguat or a pharmaceutically acceptable salt thereof administered is from about 0.2 mg to about 5 mg daily.

23. The method of claim 1, further comprising administering a third active agent comprising at least one drug selected from the group consisting of prostanoids, endothelin receptor antagonists other than ambrisentan, calcium channel blockers, diuretics, anticoagulants, a stimulator of soluble guanylate cyclase other than riociguat, oxygen and combinations thereof.

24. The method of claim 1 wherein the amount of ambrisentan or a pharmaceutically acceptable salt thereof is about 5 mg and the amount of riociguat or a pharmaceutically acceptable salt thereof is selected from the group consisting of 0.1 mg, 0.5 mg, 1 mg, 1.5 mg, 2.0 mg or 2.5 mg.

25. The method of claim 1 wherein the amount of ambrisentan or a pharmaceutically acceptable salt thereof is about 10 mg and the amount of riociguat or a pharmaceutically acceptable salt thereof is selected from the group consisting of 0.1 mg, 0.5 mg, 1 mg, 1.5 mg, 2.0 mg or 2.5 mg.

26. The method of claim 1, wherein ambrisentan or a pharmaceutically acceptable salt thereof to riociguat or a pharmaceutically acceptable salt thereof are administered once daily.

27. The method of claim 1, wherein the pulmonary hypertension comprises pulmonary arterial hypertension (PAH).

28. The method of claim 27, wherein the PAH comprises idiopathic PAH, familial PAH or PAH associated with another disease or condition.

29. The method of claim 27, wherein the PAH at baseline is of WHO Class II, III or IV.

30. A pharmaceutical formulation comprising an effective amount of ambrisentan or a pharmaceutically acceptable salt thereof and an effective amount of riociguat or a pharmaceutically acceptable salt thereof.

31. The pharmaceutical formulation of claim 30, wherein the weight ratio of ambrisentan or a pharmaceutically acceptable salt thereof to riociguat or a pharmaceutically acceptable salt thereof is in a range from about 1:1 to about 1:10.

32. The pharmaceutical formulation of claim 30, wherein the weight ratio of ambrisentan or a pharmaceutically acceptable salt thereof to riociguat or a pharmaceutically acceptable salt thereof is in a range from about 1:1 to about 100:1.

33. The pharmaceutical formulation of claim 30, wherein the weight ratio of ambrisentan or a pharmaceutically acceptable salt thereof to riociguat or a pharmaceutically acceptable salt thereof is in a range from about 1:1 to about 10:1.

34. The pharmaceutical formulation of claim 30 comprising 5 mg of ambrisentan or a pharmaceutically acceptable salt thereof in combination with 0.1 mg, 0.5 mg, 1 mg, 1.5 mg, 2.0 mg or 2.5 mg of riociguat or a pharmaceutically acceptable salt and one or more excipients.

35. The pharmaceutical formulation of claim 30 comprising 10 mg of ambrisentan or a pharmaceutically acceptable salt thereof in combination with 0.1 mg, 0.5 mg, 1 mg, 1.5 mg, 2.0 mg or 2.5 mg of riociguat or a pharmaceutically acceptable salt and one or more excipients.

36. The pharmaceutical formulation of claim 30, wherein the effectiveness of combination of ambrisentan or a pharmaceutically acceptable salt thereof to riociguat or a pharmaceutically acceptable salt thereof is at least about 25% greater than the sum of effectiveness of mono-administrations of ambrisentan or a pharmaceutically acceptable salt thereof to riociguat or a pharmaceutically acceptable salt thereof.
